(19) United States
(12) Patent Application Publication
(10) Pub. No.: US 2011/0077463 A1
(43) Pub. Date: Mar. 31, 2011

(54) OPTICAL PROBE AND ENDOSCOPE APPARATUS

(75) Inventor: Kazuhiro HIROTA, Ashigarakami-gun (JP)

(73) Assignee: FUJIFILM CORPORATION, Tokyo (JP)

(21) Appl. No.: 12/892,605

(22) Filed: Sep. 28, 2010

(30) Foreign Application Priority Data
Sep. 29, 2009 (JP) 2009-225045
Sep. 29, 2009 (JP) 2009-225046

(51) Int. Cl. A61B 1/07; A61B 1/00

(52) U.S. Cl. 600/114; 600/167

(57) ABSTRACT

According to the optical probe of an aspect of the present invention, the flexible and optically transparent partition wall separates the imaging core lumen and the guidewire lumen, and the pressure increasing/decreasing port increases/decreases the pressure inside the imaging core lumen that is at a proximal part of the imaging core lumen. It is therefore possible to advance the probe along the guidewire as far as an affected area, and by pulling back the guidewire to a handle portion to push out the imaging core to the distal end portion, it is possible to observe a distal part and obtain an image without guidewire artifacts.

### BACKGROUND OF THE INVENTION

1. Field of the Invention

The present invention relates to an optical probe and an endoscope apparatus that acquire an optical coherence tomographic image inside a body cavity, and more particularly to an optical probe and an endoscope apparatus that have a guidewire lumen through which a guidewire that aids insertion into a body cavity can be passed.

2. Description of the Related Art

Diagnostic imaging in which an optical probe is inserted into a body cavity such as a blood vessel, bile duct, pancreatic duct, stomach, esophagus or colon to obtain a tomographic image of a living organism by performing radial scanning is already being widely performed. As an example thereof, optical coherent tomography (OCT) is being utilized in which a probe that contains therein an optical fiber having an optical lens and an optical mirror attached at the distal end thereof is inserted into a body cavity, and light is radiated into the body cavity while radially scanning the optical mirror arranged on the distal end side of the optical fiber to obtain a cross-sectional image of the body cavity based on reflected light from tissue (Japanese Patent No. 4021975).

When inserting such a probe into a body cavity, generally, a guidewire is passed through a forceps opening of an endoscope and retained at an affected area before inserting the probe. The probe is then passed through as far as the affected area by being guided along the guidewire. A cross-sectional schematic diagram of a probe that has a guidewire lumen is shown in FIG. 19.

An ultrasound probe having a configuration that is comparatively similar to the above described OCT probe has also been proposed in which a guidewire lumen and an imaging core lumen that houses an imaging core that contains a sensor or the like are combined at a distal end portion to form a single lumen, to thereby enable exclusive use of a common lumen in which the guidewire and imaging core lumen have been made common (Japanese Patent No. 3367666).

A cross-sectional schematic diagram of a probe having a common lumen in which a guidewire lumen and an imaging core lumen have been made common is shown in FIG. 20 and FIG. 21. With respect to the ultrasound probe described in Japanese Patent No. 3367666, when delivering the probe to an observation portion, the insertability of the probe is improved by retracting the imaging core inside the imaging core lumen and using the common lumen as a guidewire lumen. Further, when performing observation, by retracting the guidewire as far as the guidewire lumen and feeding the imaging core as far as the common lumen of the distal end portion, it is possible to provide a cross-sectional image at the distal end portion of the probe.

In the case of OCT, in principle, the depth that a probe reaches in biological tissue is a shallow depth of 1 to 2 mm. Therefore, when a thin probe is brought into close contact with biological tissue, only a narrow range can be observed. In order to scan a wide range, it is necessary to make the probe thick. However this is not practical because the probe will be inserted from a forceps opening of an endoscope. To reconcile these problems, a method has been proposed whereby, after a probe is inserted inside the body, a balloon arranged at a distal end portion of the probe is expanded to enable scanning in a state in which the distal end portion of the probe is maintained at a fixed distance from the biological tissue (Japanese Patent Application Laid-Open No. 2000-329534).

According to Japanese Patent Application Laid-Open No. 2000-329534, technology is disclosed in which an imaging core lumen and a guidewire lumen are combined at a distal end portion, a balloon is arranged at the distal end portion of the probe, and the balloon is expanded at a time of observation.

### SUMMARY OF THE INVENTION

However, although Japanese Patent No. 4021975 discloses arranging a guidewire lumen at a front portion of an imaging core lumen, since the guidewire lumen is present at the front of the imaging core lumen, it is not possible to observe a cross section of the distal end portion. Further, since the imaging core and the guidewire are side by side at a time of observation, guidewire artifacts appear in an obtained image.

More specifically, when a guidewire lumen is provided at the front of the imaging core lumen as shown in FIG. 19, it is not possible to dispose an optical member at the distal end of the probe, and thus an image of a distal end portion can not be observed. Further, at the time of diagnosis, the physician requires that the lumen being observed is visualized at a position that is as close as possible to the distal end. Moreover, with the configuration described in Japanese Patent No. 4021975, there is the problem that guidewire artifacts are visualized in images at the time of observation, and it is also not possible to observe tissue that is at the rear of the guidewire.

Japanese Patent No. 3367666 discloses an ultrasound probe (ultrasound catheter) having a guidewire lumen and an imaging core lumen in which both lumens are combined at a distal end portion. However, when applying this technology to an optical probe, since the distal end portion of the imaging core lumen is open, a normal image cannot be obtained due to the entry of blood or body fluids or the like to the imaging core lumen.

More specifically, when a technique that combines a guidewire lumen and an imaging core lumen at a distal end portion as in the ultrasound probe described in Japanese Patent No. 3367666 is applied to an optical probe, as shown in FIG. 20 and FIG. 21, the distal end portion of the imaging core lumen is open and consequently blood or body fluids enter into the imaging core portion and a normal image cannot be obtained. Hence, practical implementation of this technique is not possible.

According to Japanese Patent Application Laid-Open No. 2000-329534, since the distal end portion of the imaging core lumen is open, a normal image cannot be obtained due to the entry of body fluids such as blood. Further, since there is a guidewire lumen inside the balloon, there is the problem that a guidewire is visualized at the time of observation.

More specifically, Japanese Patent Application Laid-Open No. 2000-329534 discloses providing a balloon at a distal end portion of a probe and expanding the balloon at a time of observation, and also discloses providing the probe with a guidewire lumen and inserting the probe by guiding the probe along the guidewire. However, with respect to the method of combining the guidewire lumen and the imaging core lumen at a distal end portion disclosed in Japanese Patent Application Laid-Open No. 2000-329534, it has to be said that the construction is inadequate because when the probe is inserted inside a body cavity, body fluids such as blood enter as far as the imaging core lumen and a normal image can not be obtained. Furthermore, when adopting a configuration that has a balloon at a distal end portion of a probe and which is provided with a guidewire lumen that is side by side with an imaging core lumen as disclosed in Japanese Patent Application Laid-Open No. 2000-329534, there are the problems that guidewire artifacts are visualized at a time of observation and tissue at the rear of the guidewire can not be visualized.

The present invention has been made in view of the above circumstances, and a first object of the invention is to provide an optical probe and an endoscope apparatus that make it possible to advance a probe as far as an affected area along a guidewire and draw back the guidewire to a handle portion to push an imaging core out to a distal end portion, to thereby enable observation of a distal part and obtainment of an image in which there are no guidewire artifacts.

A second object of the present invention is to provide an optical probe and an endoscope apparatus in which it is possible to advance a probe as far as an affected area along a guidewire, and which can obtain a tomographic image of a wide area without guidewire artifacts.

An optical probe according to a first aspect of the present invention as a first invention for achieving the first object is an optical probe that includes an optical fiber and an optical component attached to a distal end portion of the optical fiber that are provided inside a sheath to be inserted into a body cavity, and that radiates a light that is transmitted through an inside of the optical fiber towards biological tissue inside the body cavity by means of the optical component, wherein the sheath includes: an imaging core lumen that houses the optical fiber in a condition in which the optical fiber is movable forward or rearward along a longitudinal axis; a guidewire lumen that is disposed approximately parallel to a distal part of the imaging core lumen; a flexible and optically transparent partition wall that separates the imaging core lumen and the guidewire lumen; and a pressure increasing/decreasing port for increasing/decreasing a pressure inside the imaging core lumen that is provided at a proximal part of the imaging core lumen.

According to the optical probe of the first aspect, the flexible and optically transparent partition wall separates the imaging core lumen and the guidewire lumen, and the pressure increasing/decreasing port increases/decreases the pressure inside the imaging core lumen that is at a proximal part of the imaging core lumen. It is therefore possible to advance the probe along the guidewire as far as an affected area, and by pulling back the guidewire to a handle portion to push out the imaging core to the distal end portion, it is possible to observe a distal part and obtain an image without guidewire artifacts.

An optical probe of a second aspect is in accordance with the optical probe of the first aspect, wherein preferably the imaging core lumen and the guidewire lumen are arranged along the longitudinal axis at a most distal part, the imaging core lumen is sealed at a portion other than the pressure increasing/decreasing port portion, and at least when the imaging core lumen is depressurized from the pressure increasing/decreasing port, the partition wall blocks off the imaging core lumen and the most distal part is caused to function as the guidewire lumen.

An optical probe of a third aspect is in accordance with the optical probe of the first aspect, wherein preferably the imaging core lumen and the guidewire lumen are arranged along the longitudinal axis at a most distal part, the imaging core lumen is sealed at a portion other than the pressure increasing/decreasing port portion, and at least when the imaging core lumen is pressurized from the pressure increasing/decreasing port, the partition wall blocks off the guidewire lumen and the most distal part is caused to function as the imaging core lumen.

An optical probe of a fourth aspect is in accordance with the optical probe of the first aspect, wherein preferably the imaging core lumen and the guidewire lumen are arranged along the longitudinal axis at a most distal part, the imaging core lumen is sealed at a portion other than the pressure increasing/decreasing port portion, and when the imaging core lumen is depressurized from the pressure increasing/decreasing port, the partition wall blocks off the imaging core lumen and the most distal part is caused to function as the guidewire lumen, and when the imaging core lumen is pressurized from the pressure increasing/decreasing port, the partition wall blocks off the guidewire lumen and the most distal part is caused to function as the imaging core lumen.

An optical probe of a fifth aspect is in accordance with an optical probe of any one of the first to fourth aspects, wherein preferably the optical fiber is arranged inside a drive shaft that rotationally drives, and an inside of the body cavity is radially scanned by rotationally driving the optical component.

An optical probe of a sixth aspect is in accordance with the optical probe of the fifth aspect, wherein preferably the drive shaft is movable along the longitudinal axis, and the inside of the body cavity is spirally scanned by driving the optical component rotationally and in an axial direction.

An optical probe of a seventh aspect is in accordance with an optical probe of any one of the first to sixth aspects, wherein preferably the optical component includes a ball lens having a reflective surface that bends, at approximately a right angle, a travelling direction of light that is transmitted through the inside of the optical fiber.

An optical probe of an eighth aspect is in accordance with an optical probe of any one of the first to seventh aspects, wherein preferably the optical fiber transmits a wavelength-sweeping laser beam.

An endoscope apparatus of a ninth aspect includes an optical probe according to any one of the first to eighth aspects, wherein the sheath of the optical probe is inserted through a treatment instrument channel of an endoscope.

An optical probe according to a tenth aspect as a second invention for achieving the second object is an optical probe that includes an optical fiber and an optical component attached to a distal end portion of the optical fiber that are provided inside a sheath to be inserted into a body cavity, and that radiates a light that is transmitted through an inside of the optical fiber towards biological tissue inside the body cavity by means of the optical component, wherein the sheath includes: an imaging core lumen that houses the optical fiber in a condition in which the optical fiber is movable forward or rearward along a longitudinal axis; a guidewire lumen that is disposed approximately parallel to a distal part of the imaging core lumen; a balloon that is arranged so as to cover an outer side of the guidewire lumen and the imaging core lumen and that is connected at one portion to the imaging core lumen; a flexible and optically transparent partition wall that separates the imaging core lumen and the guidewire lumen; and a pressure increasing/decreasing port for increasing/decreasing a pressure inside the imaging core lumen that is provided at a proximal part of the imaging core lumen; and wherein: the imaging core lumen, the guidewire lumen, and the balloon are disposed along the longitudinal axis at a most distal part; the imaging core lumen is connected to the balloon and is sealed at a distal part, and depressurizing the imaging core lumen causes the partition wall to block off the imaging core lumen and causes the most distal part to function as the guidewire lumen and also deflates the balloon, and pressurizing the imaging core lumen causes the partition wall to block off the guidewire lumen and causes the most distal part to function as the imaging core lumen and expands the balloon.

According to the optical probe of the tenth aspect, the imaging core lumen is connected to the balloon and is sealed at the distal part so that depressurizing the imaging core lumen causes the partition wall to block off the imaging core lumen and causes the most distal part to function as the guidewire lumen and also deflates the balloon, and pressurizing the imaging core lumen causes the partition wall to block off the guidewire lumen and causes the most distal part to function as the imaging core lumen and expands the balloon. It is thereby possible to advance the probe along the guidewire as far as an affected area, and also obtain a tomographic image of a wide area without guidewire artifacts.

An optical probe of an eleventh aspect is in accordance with the optical probe of the tenth aspect, wherein preferably the optical fiber is arranged inside a drive shaft that rotationally drives, and an inside of the body cavity is radially scanned by rotationally driving the optical component.

An optical probe of a twelfth aspect is in accordance with the optical probe of the tenth aspect, wherein preferably the drive shaft is also movable along the axial direction, and an inside of the body cavity is spirally scanned by driving the optical component rotationally and forward or rearward in an axial direction driving range.

An optical probe of a thirteenth aspect is in accordance with the optical probe of any one of the tenth to twelfth aspects, wherein preferably the optical component includes a ball lens having a reflective surface that bends, at approximately a right angle, a travelling direction of the light that is transmitted through the inside of the optical fiber.

An optical probe of a fourteenth aspect is in accordance with the optical probe of any one of the tenth to thirteenth aspects, wherein preferably the optical fiber transmits a wavelength-sweeping laser beam to the inside of the body cavity.

An optical probe of a fifteenth aspect is in accordance with the optical probe of any one of the tenth to fourteenth aspects, wherein preferably a diameter at both ends of the balloon is larger than a diameter of a center part thereof.

An optical probe of a sixteenth aspect is in accordance with the optical probe of any one of the tenth to fifteenth aspects, wherein preferably the imaging core lumen is connected to a plurality of balloons.

An optical probe of a seventeenth aspect is in accordance with the optical probe of the twelfth aspect, wherein preferably the plurality of balloons are arranged at front and rear of the axial direction driving range.

An endoscope apparatus of an eighteenth aspect includes an optical probe according to any one of the tenth to seventeenth aspects, wherein the sheath of the optical probe is inserted through a treatment instrument channel of an endoscope.

An optical probe according to a nineteenth aspect is an optical probe that includes an optical fiber and an optical component attached to a distal end portion of the optical fiber that are provided inside a sheath to be inserted into a body cavity, and that radiates a light that is transmitted through an inside of the optical fiber towards biological tissue inside the body cavity by means of the optical component, wherein the sheath includes: an imaging core lumen that houses the optical fiber having the optical component along a longitudinal axis; a balloon that is disposed so as to cover an outer side of the imaging core lumen; and a port for expanding/contracting the balloon by pressurization/depressurization that is at a proximal part of the sheath; and wherein: the optical component includes a focus adjustment mechanism; and a diameter of the balloon can be varied by a pressure that is applied.

An optical probe of a twentieth aspect is in accordance with the optical probe of the nineteenth aspect, wherein preferably a focus obtained by the focus adjustment mechanism is controlled in accordance with a diameter of the balloon.

An optical probe of a twenty-first aspect is in accordance with the optical probe of the nineteenth or twentieth aspect, wherein preferably the sheath further includes a guidewire lumen that is disposed approximately parallel to a distal part of the imaging core lumen.

An optical probe of a twenty-second aspect is in accordance with the optical probe of any one of the nineteenth to twenty-first aspects, wherein preferably: the balloon is arranged so as to cover an outer side of the guidewire lumen and the imaging core lumen and is connected at one portion with the imaging core lumen; the imaging core lumen, the guidewire lumen, and the balloon are coaxially disposed at a most distal part; the port is connected to the imaging core lumen; the sheath further includes a flexible and optically transparent partition wall that separates the imaging core lumen and the guidewire lumen; by depressurizing the imaging core lumen, the partition wall is caused to block off the imaging core lumen, the most distal part is caused to function as a guidewire lumen and the balloon is deflated; and by pressurizing the imaging core lumen, the partition wall is caused to block off the guidewire lumen, the most distal part is caused to function as an imaging core lumen, and the balloon is expanded.

As described above, according to the first invention there are the advantages that it is possible to advance a probe along a guidewire as far as an affected area, and by pulling back the guidewire to a handle portion and pushing out the imaging core to the distal end portion, it is possible to perform observation of a distal part and obtain an image without any guidewire artifacts.

Further, according to the second invention, there is the advantage that it is possible to advance a probe along a guidewire to an affected area, and obtain a tomographic image of a wide area without any guidewire artifacts.

### BRIEF DESCRIPTION OF THE DRAWINGS

FIG. 1 is a block diagram that shows the internal configuration of an OCT probe and an OCT processor according to a first embodiment;

FIG. 2 is a sectional view that shows the configuration of an optical rotary joint that connects a rotation-side optical fiber FB1 shown in FIG. 1;

FIG. 3 is a sectional view of a sheath portion (when a flexible partition wall member is contracted) of an OCT probe according to the first embodiment;

FIG. 4 is a view that shows a cross section along line A-A in FIG. 3;

FIG. 5 is a view that shows a cross section along line B-B in FIG. 3;

FIG. 6 is a sectional view of a sheath portion (when a flexible partition wall member is expanded) of an OCT probe according to the first embodiment;

FIG. 7 is a view that shows a cross section along line C-C in FIG. 6;

FIG. 8 is a sectional view of a sheath portion (when a flexible partition wall member is contracted) of an OCT probe according to a second embodiment;

FIG. 9 is a view that shows a cross section along line A-A in FIG. 8;

FIG. 10 is a view that shows a cross section along line B-B in FIG. 8;

FIG. 11 is a view that shows a cross section along line C-C in FIG. 8;

FIG. 12 is a sectional view of a sheath portion (when a flexible partition wall member is expanded) of an OCT probe according to a second embodiment;

FIG. 13 is a view that shows a cross section along line D-D in FIG. 12;

FIG. 14 is a cross-sectional schematic diagram of a modification example 1 of the second embodiment;

FIG. 15 is a cross-sectional schematic diagram of a modification example 2 of the second embodiment;

FIG. 16 is a cross-sectional schematic diagram of a modification example 3 of the second embodiment;

FIG. 17 is a view that shows an optical lens system at a distal end portion of an imaging core shown in FIG. 16;

FIG. 18 is a view that illustrates a diagnostic imaging apparatus in which an OCT probe is used together with an endoscope apparatus to which the OCT probe can be applied;

FIG. 19 is a cross-sectional schematic diagram of a probe that has a conventional guidewire lumen;

FIG. 20 is a first cross-sectional schematic diagram of a conventional probe having a common lumen in which a guidewire lumen and an imaging core lumen are made common; and

FIG. 21 is a second cross-sectional schematic diagram of a conventional probe having a common lumen in which a guidewire lumen and an imaging core lumen are made common.

### DETAILED DESCRIPTION OF THE PREFERRED EMBODIMENTS

Hereunder, respective embodiments according to the first and second inventions are described in detail with reference to the attached drawings.

### First Embodiment

First, an embodiment (first embodiment) relating to the first invention is described.

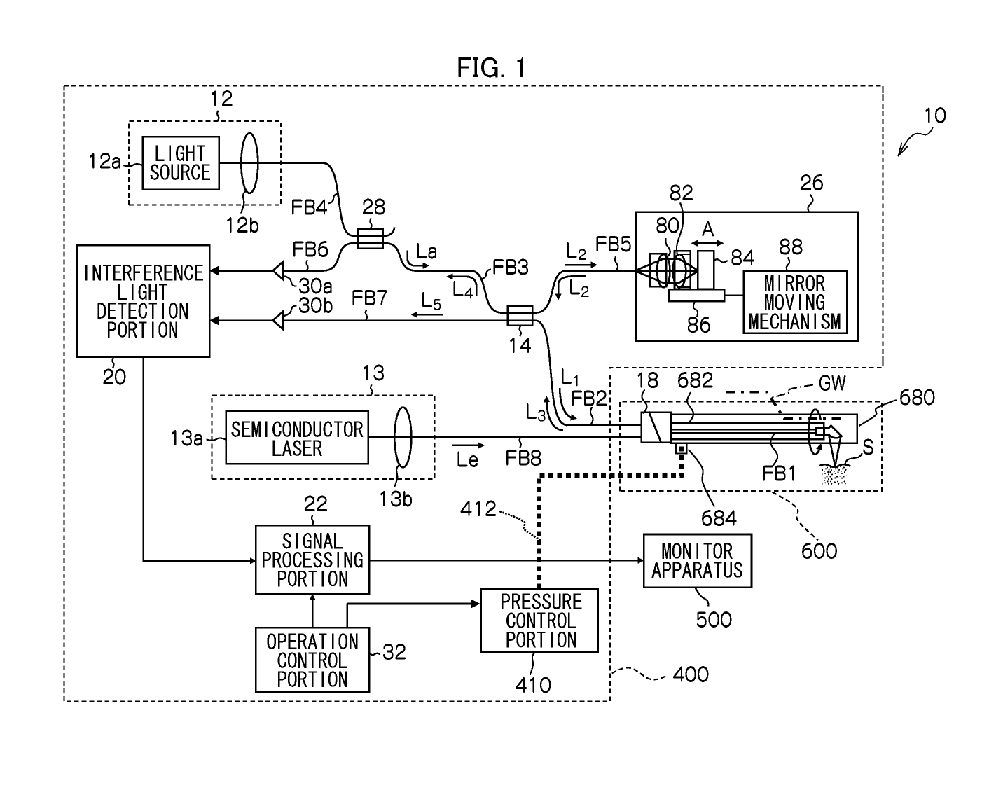

As shown in FIG. 1, an OCT probe 600 and an OCT processor 400 of the present embodiment are used for acquiring an optical tomographic image of an object to be measured by using the optical coherence tomography (OCT) technique.

### [OCT Processor]

The OCT processor 400 includes a first light source (a first light source unit) 12 that emits a light La for measurement; an optical fiber coupler (a branching/multiplexing portion) 14 that branches the light La emitted from the first light source 12 into a measurement light (a first light flux) L1 and a reference light L2, and multiplexes a returning light L3 from an object to be measured S as a subject and the reference light L2, thereby generating an interference light L4; the OCT probe 600 including a rotation-side optical fiber FB1 that guides the measurement light L1 that is branched at the optical fiber coupler 14 to the object to be measured and guides the returning light L3 from the object to be measured; a fixed-side optical fiber FB2 that guides the measurement light L1 to the rotation-side optical fiber FB1 and also guides the returning light L3 that has been guided by the rotation-side optical fiber FB1; an optical connector 18 that rotatably connects the rotation-side optical fiber FB1 to the fixed-side optical fiber FB2 and transmits the measurement light L1 and the returning light L3; an interference light detection portion 20 that detects the interference light L4 generated by the optical fiber coupler 14 as an interference signal; and a processing portion 22 that processes the interference signal detected by the interference light detection portion 20 to acquire optical structure information. Further, the OCT processor 400 displays an image based on the optical structure information acquired by the processing portion 22 on a monitor apparatus 500.

The OCT processor 400 also includes a second light source (a second light source unit) 13 that emits an aiming light (a second light flux) Le for indicating a mark for measurement; an optical path length adjustment portion 26 that adjusts an optical path length of the reference light L2; an optical fiber coupler 28 that branches the light La emitted from the first light source 12; detection portions 30a and 30b that detect returning lights L4 and L5 that are multiplexed at the optical fiber coupler 14; and an operation control portion 32 that inputs various conditions to the processing portion 22 and changes settings and the like thereof.

In the OCT processor 400 shown in FIG. 1, various optical fibers FB (FB3, FB4, FB5, FB6, FB7, FB8 and the like) including the rotation-side optical fiber FB1 and the fixed-side optical fiber FB2 are used as an optical path for guiding and transmitting various lights including the emission light La, the aiming light Le, the measurement light L1, the reference light L2, and returning light L3 between components such as each optical device.

The first light source 12 emits a laser beam or low coherence light for OCT measurement. The first light source 12 is a light source that emits the laser light La that is centered on, for example, a wavelength of 1.3 μm while sweeping the laser light La at a fixed cycle. The first light source 12 includes a light source 12a that emits a laser beam or a low-coherence light La, and a lens 12b that collects the light La emitted from the light source 12a. As will be described in detail later, the light La emitted from the first light source 12 is divided into the measurement light L1 and the reference light L2 by the optical fiber coupler 14 via the optical fibers FB4 and FB3, and the measurement light L1 is input to the optical connector 18.

The second light source 13 emits visible light as the aiming light Le for facilitating confirmation of a measurement site. For example, a red semiconductor laser beam with a wavelength of 0.66 μm, a He—Ne laser beam with a wavelength of 0.63 μm, and a blue semiconductor laser beam with a wavelength of 0.405 μm and the like can be used. The second light source 13 includes a semiconductor laser 13a that emits, for example, a red, blue, or green laser beam, and a lens 13b that collects the aiming light Le emitted from the semiconductor laser 13a. The aiming light Le emitted from the second light source 13 is input to the optical connector 18 via the optical fiber FB8.

The measurement light L1 and the aiming light Le are multiplexed at the optical connector 18, and the multiplexed light is guided to the rotation-side optical fiber FB1 in the OCT probe 600.

The optical fiber coupler (the branching/multiplexing portion) 14 is composed of, for example, 2×2 optical fiber couplers, and is optically connected to the fixed-side optical fiber FB2, optical fiber FB3, optical fiber FB5, and optical fiber FB7, respectively.

The optical fiber coupler 14 divides the light La entering via the optical fibers FB4 and FB3 from the first light source 12 into the measurement light (the first light flux) L1 and the reference light L2, and causes the measurement light L1 to enter the fixed-side optical fiber FB2 and causes the reference light L2 to enter the optical fiber FB5.

Further, the optical fiber coupler 14 multiplexes the light L2 that enters the optical fiber FB5 and which is returned through the optical fiber FB5 after being subjected to a frequency shift and an optical path length adjustment by the optical path length adjustment portion 26 that is described later, and a light L3 that is acquired by the OCT probe 600 as will be described later and is guided from the fixed-side optical fiber FB2, and emits the multiplexed light to the optical fiber FB3 (FB6) and the optical fiber FB7.

The OCT probe 600 is connected to the fixed-side optical fiber FB2 via the optical connector 18. The measurement light L1 that is multiplexed with the aiming light Le is caused to enter the rotation-side optical fiber FB1 from the fixed-side optical fiber FB2 via the optical connector 18. The incident measurement light L1 that has been multiplexed with the aiming light Le is transmitted by the rotation-side optical fiber FB1 to illuminate the object to be measured S. The returning light L3 from the object to be measured S is acquired. The acquired returning light L3 is transmitted by the rotation-side optical fiber FB1, and is emitted to the fixed-side optical fiber FB2 via the optical connector 18.

The optical connector 18 multiplexes the measurement light (the first light flux) L1 and the aiming light (the second light flux) Le.

The interference light detection portion 20 is connected to the optical fiber FB6 and the optical fiber FB7. The interference light detection portion 20 detects as an interference signal the interference lights L4 and L5 that are generated by multiplexing the reference light L2 and the returning light L3 at the optical fiber coupler 14.

The OCT processor 400 includes a detecting element 30a that is provided on the optical fiber FB6 that branches from the optical fiber coupler 28 and detects the light intensity of the interference light L4, and a detecting element 30b that detects the light intensity of the interference light L5 on the optical path of the optical fiber FB7.

The interference light detection portion 20 detects the intensity of reflected light (or backward scattered light) at each depth position of the object to be measured S by subjecting the interference light L4 detected from the optical fiber FB6 and the interference light L5 detected from the optical fiber FB7 to Fourier transformation based on the detection results of the detecting element 30a and the detecting element 30b.

The processing portion 22 detects a region where the OCT probe 600 and the object to be measured S are contacting at a measurement position based on an interference signal extracted by the interference light detection portion 20. More precisely, the processing portion 22 detects a region where a surface of a probe outer tube (described later) of the OCT probe 600 and the surface of the object to be measured S are considered to be in contact. Further, the processing portion 22 acquires optical structure information based on the interference signal detected by the interference light detection portion 20, generates an optical three-dimensional image based on the acquired optical structure information, and outputs an image obtained by performing various kinds of processing with respect to the optical three-dimensional image to the monitor apparatus 500. The detailed configuration of the processing portion 22 is described later.

The optical path length adjustment portion 26 is arranged on the emission side of the reference light L2 of the optical fiber FB5 (more specifically, an end portion on the opposite side to the optical fiber coupler 14 of the optical fiber FB5).

The optical path length adjustment portion 26 has a first optical lens 80 that shapes the light emitted from the optical fiber FB5 into collimated light, a second optical lens 82 that collects the light shaped into the collimated light by the first optical lens 80, a reflection mirror 84 that reflects the light collected by the second optical lens 82, a base 86 that supports the second optical lens 82 and the reflection mirror 84, and a mirror moving mechanism 88 that moves the base 86 in a direction parallel to an optical axis direction. The optical path length adjustment portion 26 adjusts the optical path length of the reference light L2 by changing a distance between the first optical lens 80 and the second optical lens 82.

The first optical lens 80 shapes the reference light L2 emitted from a core of the optical fiber FB5 into collimated light, and also collects the reference light L2 reflected by the reflection mirror 84 into the core of the optical fiber FB5.

The second optical lens 82 collects the reference light L2 shaped into the collimated light by the first optical lens 80 on the reflection mirror 84, and also shapes the reference light L2 reflected by the reflection mirror 84 into collimated light. Thus, a confocal optical system is formed by the first optical lens 80 and the second optical lens 82.

Further, the reflection mirror 84 is arranged at the focal point of the light that is collected by the second optical lens 82, and reflects the reference light L2 collected by the second optical lens 82.

Thus, the reference light L2 emitted from the optical fiber FB5 is shaped into collimated light by the first optical lens 80, and is collected onto the reflection mirror 84 by the second optical lens 82. Thereafter, the reference light L2 reflected by the reflection mirror 84 is shaped into collimated light by the second optical lens 82, and is collected into the core of the optical fiber FB5 by the first optical lens 80.

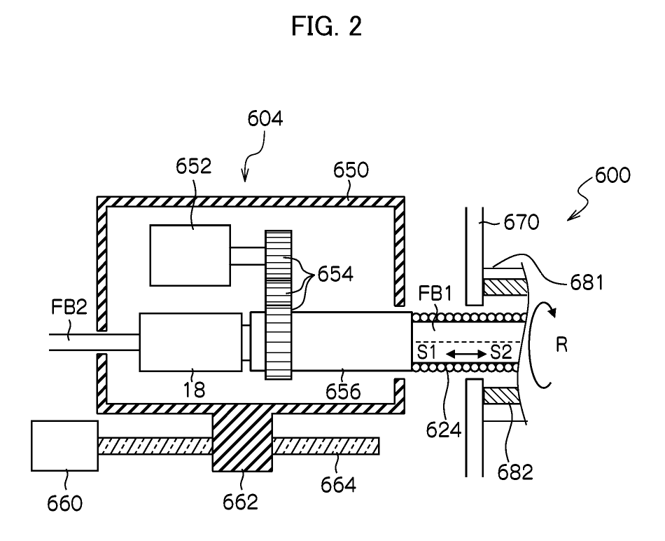

The base 86 fixes the second optical lens 82 and the reflection mirror 84. The mirror moving mechanism 88 moves the base 86 in the optical axis direction (the direction of an arrow A in FIG. 2) of the first optical lens 80.

The mirror moving mechanism 88 can change the distance between the first optical lens 80 and the second optical lens 82 by moving the base 86 in the direction of the arrow A, and thus the optical path length of the reference light L2 can be adjusted.

The operation control portion 32 includes an input device such as a keyboard and a mouse, and a control device that manages various conditions based on input information. The operation control portion 32 is connected to the processing portion 22. The operation control portion 32 inputs, sets, and changes various processing conditions or the like in the processing portion 22 based on an instruction of the operator input from the input device.

The operation screen of the operation control portion 32 may be displayed on the monitor apparatus 500, or on a separately provided display portion. The operation control portion 32 may also perform operation control and set various conditions of the first light source 12, the second light source 13, the optical connector 18, the interference light detection portion 20, the optical path length, and the detection portions 30a and 30b.

As shown in FIG. 2, the rotation-side optical fiber FB1 and fixed-side optical fiber FB2 are connected by the optical connector 18, so that the rotation-side optical fiber FB1 and fixed-side optical fiber FB2 are optically connected in a state in which rotation of the rotation-side optical fiber FB1 is not transmitted to the fixed-side optical fiber FB2. Further, the rotation-side optical fiber FB1 is arranged in a state in which the rotation-side optical fiber FB1 is rotatable with respect to an imaging core lumen 681 and is movable in the axial direction of the imaging core lumen 681.

A torque transmitting coil 624 is fixed to the outer circumference of the rotation-side optical fiber FB1. The rotation-side optical fiber FB1 and the torque transmitting coil 624 are connected to an optical rotary joint of the optical connector 18.

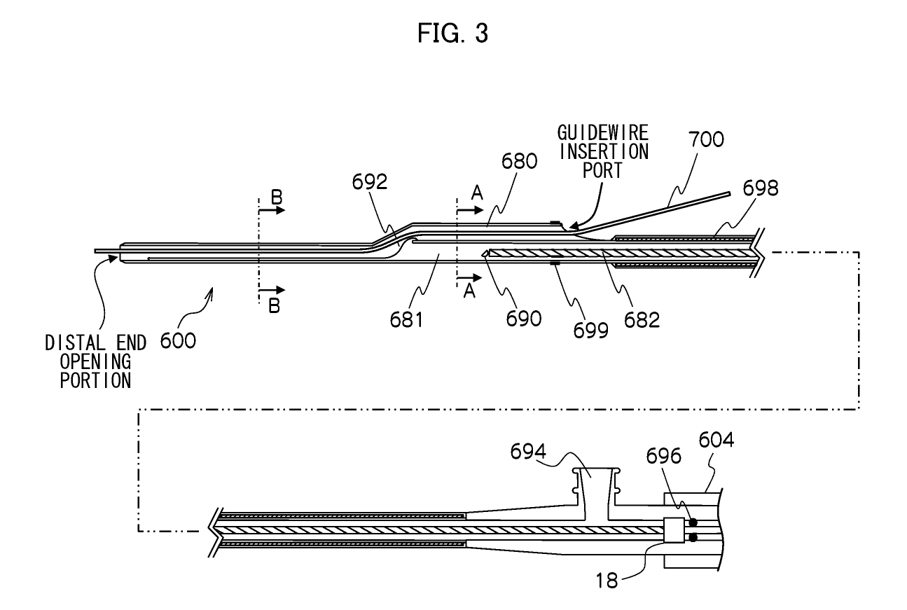

The rotation-side optical fiber FB1, the torque transmitting coil 624, and the ball lens (optical lens) 690 (see FIG. 3) are arranged to be movable in the direction of an arrow S1 (forceps opening direction) and the direction of an arrow S2 (direction of distal end of imaging core lumen 681) inside the imaging core lumen 681 by a back and forth driving portion provided in the optical connector 18 as described later.

The imaging core lumen 681 is fixed by a fixing member 670. The rotation-side optical fiber FB1 and the torque transmitting coil 624 are connected to a rotary cylinder 656. The rotary cylinder 656 is configured so as to rotate via a gear 654 in accordance with rotation of a motor 652. The rotary cylinder 656 is connected to the optical rotary joint of the optical connector 18. The measurement light L1 and the returning light L3 are transmitted between the rotation-side optical fiber FB1 and the fixed-side optical fiber FB2 via the optical connector 18.

A frame 650 that incorporates therein the optical connector 18, the motor 652, the gear 654, and the rotary cylinder 656 includes a support member 662. The support member 662 has an unshown screw hole. A ball screw for back and forth movement 664 meshes with the frame 650 at a screw hole (unshown) of the support member 662. A motor 660 is connected to the ball screw for back and forth movement 664, so that a back and forth driving portion as a back and forth movement device is composed by the screw hole, the ball screw for back and forth movement 664, the motor 660, and the like. Accordingly, the back and forth driving portion of the optical rotary joint of the optical connector 18 drives the frame 650 so as to move forward or backward by rotational driving of the motor 660. As a result, the rotation-side optical fiber FB1, the torque transmitting coil 624, the fixing member 670, and the ball lens 690 can be moved in the directions of S1 and S2 in FIG. 2.

The motor 660 drives forward/backward at a predetermined pitch, for example, at intervals of 1 mm. For each predetermined pitch, the motor 652 rotates the rotation-side optical fiber FB1, the torque transmitting coil 624, and the ball lens 690 one time, to thereby illuminate the object to be measured S by radially scanning the measurement light L1.

The OCT probe 600 has the above configuration. The rotation-side optical fiber FB1 and the torque transmitting coil 624 are rotated in the direction of an arrow R in FIG. 2 by the optical rotary joint of the optical connector 18. The OCT probe 600 thereby illuminates the object to be measured S with the measurement light L1 emitted from the ball lens 690 while radially scanning in the direction of the arrow R (the circumferential direction of the imaging core lumen 681), and acquires the returning light L3.

Accordingly, a desired portion of the object to be measured S can be accurately captured over the entire periphery of the imaging core lumen 681 in the circumferential direction, and the returning light L3 reflected from the object to be measured S can be acquired.

In a case of acquiring a plurality of items of optical structure information for generating an optical three-dimensional image, the ball lens 690 is moved in the direction of the arrow S1 in FIG. 2 to one end of a moveable range by the back and forth driving portion of the optical rotary joint of the optical connector 18. The ball lens 690 then moves in the direction of the arrow S2 by a predetermined distance at a time until reaching the other end of the movable range while acquiring the optical structure information comprising tomographic images, or alternately acquires the optical structure information and moves a predetermined distance in the direction of S2 in FIG. 2 until reaching the other end of the movable range.

It is thus possible to acquire a plurality of items of optical structure information over a desired area of the object to be measured S, and obtain an optical three-dimensional image based on the acquired plurality of items of optical structure information.

More specifically, the OCT probe 600 acquires optical structure information in the depth direction (first direction) of the object to be measured S by means of an interference signal, and radially scans in the arrow R direction (the circumferential direction of the imaging core lumen 681) in FIG. 2 with respect to the object to be measured S to thereby enable acquisition of optical structure information on a scanning plane comprising the depth direction of the object to be measured S (first direction) and a direction that is approximately perpendicular to the depth direction (second direction). Further, by moving the scanning plane along a direction (third direction) that is approximately perpendicular to the scanning plane, a plurality of items of optical structure information for generating an optical three-dimensional image can be acquired.

As shown in FIG. 3, the configuration of the imaging core of the OCT probe 600 that includes the drive shaft 682, the optical fiber FB1, and the torque transmitting coil 624 inside the drive shaft 682, and the ball lens 690 provided at the distal end of the optical fiber FB1 is the same as that of the conventional optical probe. However, in the imaging core of the present embodiment, the optical fiber FB1 having the ball lens 690 at the distal end thereof inside the drive shaft 682 is rotated by rotating the torque transmitting coil 624 disposed on the outside thereof to thereby perform radial scanning. Further, the drive shaft 682 also simultaneously performs axial direction scanning by means of a direct-acting mechanism provided at a handle portion, to thereby perform spiral scanning (see FIG. 2).

A sheath portion of the OCT probe 600 is a principal component relating to the present embodiment. The operator-hand side of the sheath portion of the OCT probe 600 is formed as a braid tube in which a metal mesh 698 is provided as an inner layer, and the internal cavity of the sheath portion can be secured even when a raising mechanism of the forceps channel of the endoscope is operated.

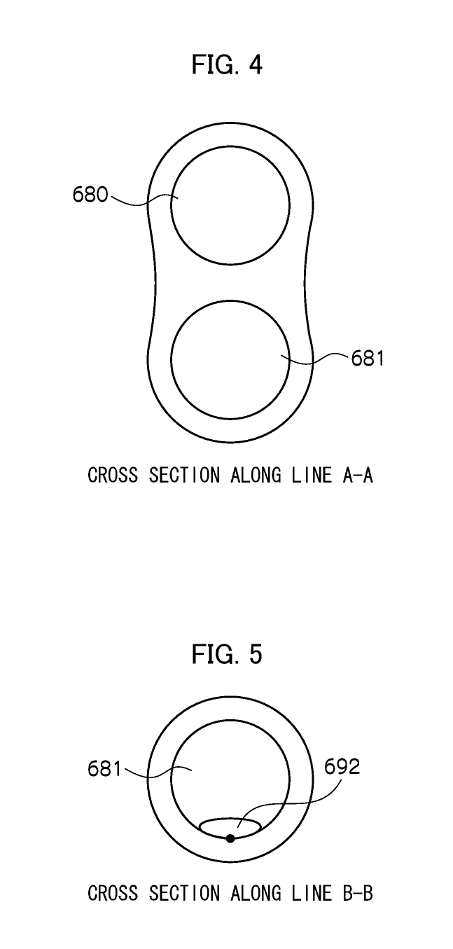

The sheath portion of the OCT probe 600 includes the imaging core lumen 681 that houses the imaging core therein (extending) along the longitudinal axis of the sheath portion of the OCT probe 600, and the guidewire lumen 680 that is disposed approximately parallel to the distal part of the imaging core lumen 681 (see FIG. 4 that shows a cross section along line A-A in FIG. 3). The two lumens 680 and 681 are connected in a separated state by a tubular partition wall member 692 comprising an optically transparent and flexible material such as, for example, silicone rubber at the distal end portion. Although silicone rubber is mentioned as an example of the optically transparent and flexible material, the material is not limited thereto, and another material such as latex rubber, nylon, or PET may be used.

As shown in FIG. 5 that shows a cross section along line B-B in FIG. 3, the imaging core lumen 681 is linearly fixed to the partition wall member 692 at a place on the bottom side, and a distal side thereof is sealed. A pressurizing/depressurizing port 694 is provided in the handle portion, and although not illustrated in the drawings, by connecting a syringe with a lock or an indeflator, the pressure inside the imaging core lumen 681 can be increased or decreased.

Hereunder, operations when inserting the probe 600 into an affected area are described. The imaging core lumen 681 is depressurized by connecting a syringe with a lock (unshown) to the pressure increasing/decreasing port 694 and drawing in air using the syringe. At that time, the flexible partition wall member 692 contracts so that the capacity of the imaging core lumen 681 becomes the minimum capacity, and the guidewire lumen 680 is opened as far as the distal end portion. Therefore, by previously passing the end of the guidewire 700 that is inserted as far as the affected area through the guidewire lumen 680, and pushing the OCT probe 600 in along the guidewire 700, the OCT probe 600 can be easily pushed forward as far as the affected area.

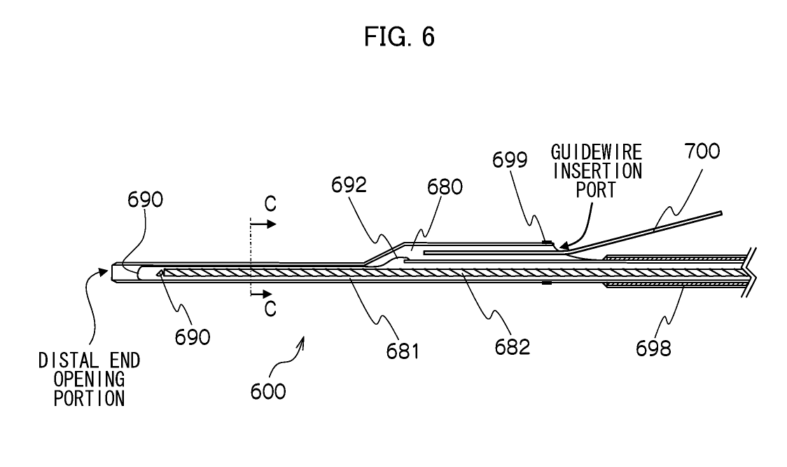

Next, operations at the time of observation are described using FIG. 6. In a state in which the OCT probe 600 is retained at the affected area, the guidewire 700 is drawn in as far as the proximal part of the guidewire lumen 680. At this time, the operations are performed so as not to extract the guidewire 700 from the guidewire lumen 680 while checking a contrast marker 699 on a fluoroscopic image.

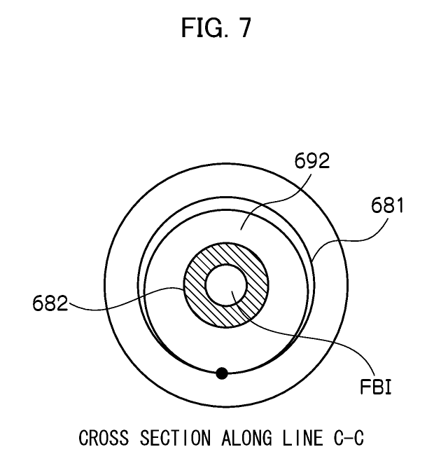

Next, by pressurizing the imaging core lumen 681 using the syringe, as shown in FIG. 7 that is a cross-section along line C-C in FIG. 6, a space of the imaging core lumen 681 is formed as far as the distal end portion thereof. In this state, observation is enabled by advancing the drive shaft 682 as far as the frontmost portion. Next, radial scanning is performed by rotating the drive shaft 682, and a spiral operation is enabled by simultaneously scanning at a constant speed in the axial direction, so that three-dimensional tomographic data of the body cavity can be acquired.

According to the OCT probe 600 of the present embodiment as described above, the imaging core is advanced as far as the distal end portion to enable observation of a cross section at the distal end portion, and since the guidewire is also drawn back to the operator side of the observation surface at the time of observation, artifacts are not generated. Further, since the imaging core lumen distal end portion is sealed, blood or the like does not enter the imaging core lumen and thus an accurate image is obtained.

Consequently, according to the present embodiment, it is possible to advance a probe as far as an affected area along a guidewire, and also to pull back the guidewire to a handle portion and push out the imaging core to the distal end portion. It is thereby possible to observe a distal part and obtain images that have no guidewire artifacts.

### Second Embodiment

Next, an embodiment (second embodiment) relating to a second invention is described. Hereunder, a description regarding portions that are common with the first embodiment is omitted, and the description centers on characteristic portions of the present embodiment.

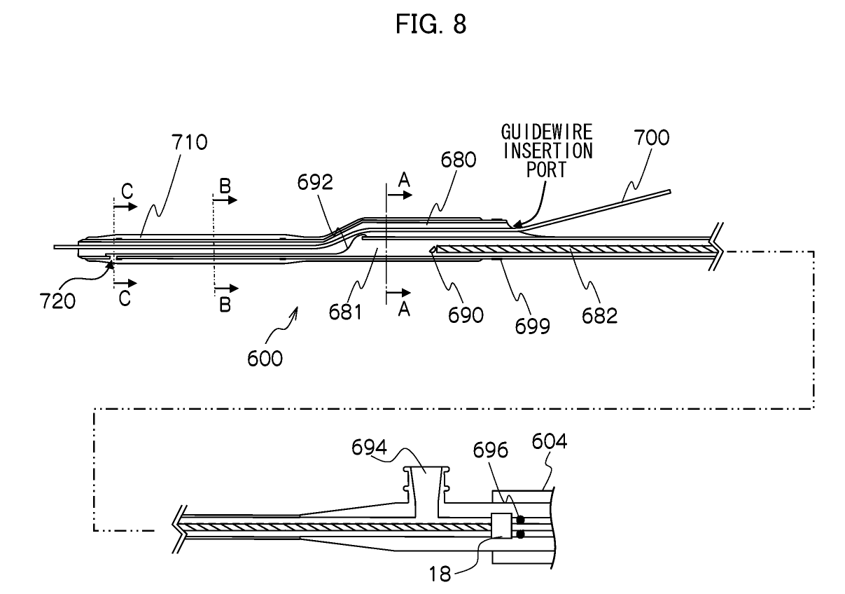

FIG. 8 is a sectional view that illustrates the sheath portion of the OCT probe according to the second embodiment. In this connection, in FIG. 8 and FIGS. 9 to 16 that are described later, components that are the same as or similar to components of the first embodiment (FIG. 3 to FIG. 5) are designated by the same reference numerals.

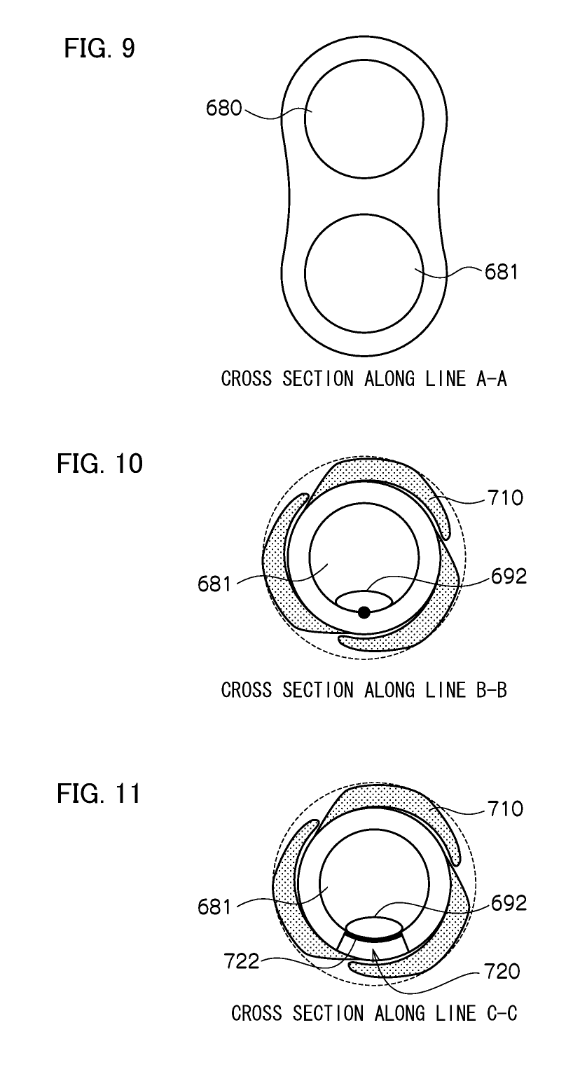

The sheath portion of the OCT probe 600 is a principal component according to the second embodiment. As shown in FIG. 8, the sheath portion of the OCT probe 600 according to the second embodiment includes the imaging core lumen 681 that houses the imaging core therein (extending) along the longitudinal axis of the sheath portion of the OCT probe 600, the guidewire lumen 680 that is disposed approximately parallel to the distal part of the imaging core lumen 681, and a balloon 710 that is disposed at the distal end portion in a condition in which the balloon 710 is folded around the circumference thereof (see FIG. 9 that shows a cross section along line A-A in FIG. 8). The two lumens 680 and 681 are connected in a separated state by a tubular partition wall member 692 comprising an optically transparent and flexible material at the distal end portion.

As shown in FIG. 10 that shows a cross section along line B-B in FIG. 8, the imaging core lumen 681 is linearly fixed to the partition wall member 692 at a place on the bottom side thereof, and is connected to the balloon 710 by a small hole (communicating hole) 720 on the distal side thereof. As shown in FIG. 11 that shows a cross section along line C-C in FIG. 8, the imaging core lumen 681 is fixed in a watertight manner at the circumference of the small hole 720 by the partition wall member 692 and an adhesive 722 in a state in which communication between the imaging core lumen 681 and the balloon 710 by means of the small hole 720 is secured.

The pressurizing/depressurizing port 694 is provided at the handle portion. Although not illustrated in the drawings, by connecting a syringe with a lock or an indeflator, the pressure inside the imaging core lumen 681 can be increased or decreased.

Hereunder, operations when inserting the OCT probe 600 into an affected area are described. The imaging core lumen 681 is depressurized by connecting a syringe with a lock (unshown) to the pressure increasing/decreasing port 694 and drawing in air using the syringe. At that time, the flexible partition wall member 692 contracts such that the capacity of the imaging core lumen 681 becomes the minimum capacity, and the guidewire lumen 680 is opened as far as the distal end portion. Therefore, by previously passing the end of the guidewire 700 that is inserted as far as the affected area through the guidewire lumen 680, and pushing in the OCT probe 600 along the guidewire 700, the OCT probe 600 can easily be pushed forward as far as the affected area.

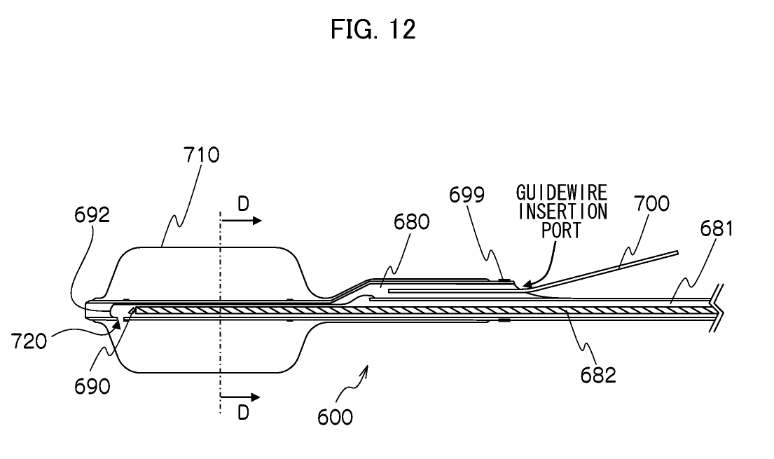

Next, operations at the time of observation are described using FIG. 12. In a state in which the OCT probe 600 is retained at the affected area, the guidewire 700 is drawn in as far as the proximal part of the guidewire lumen 680. At this time, the operations are performed so as not to extract the guidewire 700 from the guidewire lumen 680 while checking the contrast marker 699 on a fluoroscopic image.

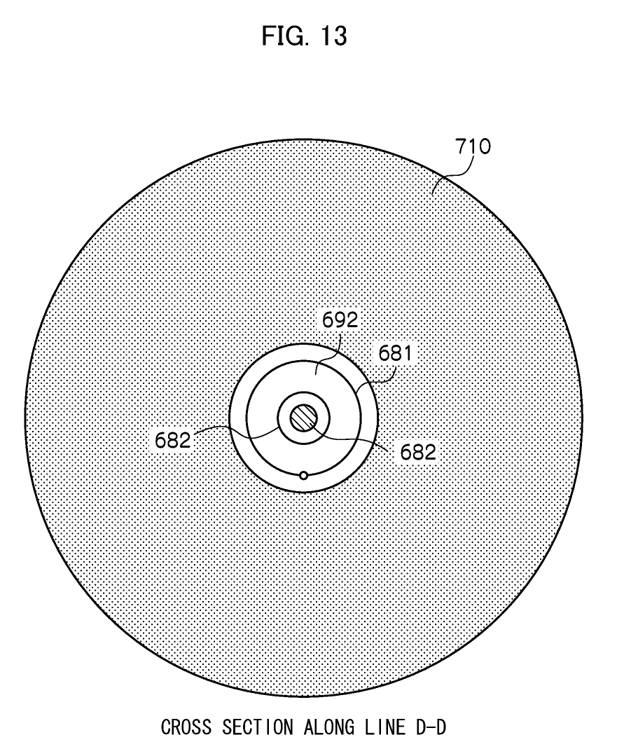

Next, by pressurizing the imaging core lumen 681 using the syringe, as shown in FIG. 13 that shows a cross section along line D-D in FIG. 12, the balloon 710 is expanded so that a fixed interval is kept between the imaging core and the observation site. At this time, the space of the imaging core lumen 681 is formed as far as the distal end portion. In this state, observation is enabled by advancing the drive shaft 682 as far as the frontmost portion. Next, radial scanning is performed by rotating the drive shaft 682, and a spiral operation is enabled by simultaneously scanning at a constant speed in the axial direction, so that three-dimensional tomographic data of the body cavity can be acquired.

According to the present embodiment as described above, it is possible to advance the probe as far as an affected area along the guidewire 700, and to obtain tomographic images of a wide area without artifacts of the guidewire 700 by expanding the balloon 710.

Next, modification examples of the second embodiment are described.

### Modification Example 1

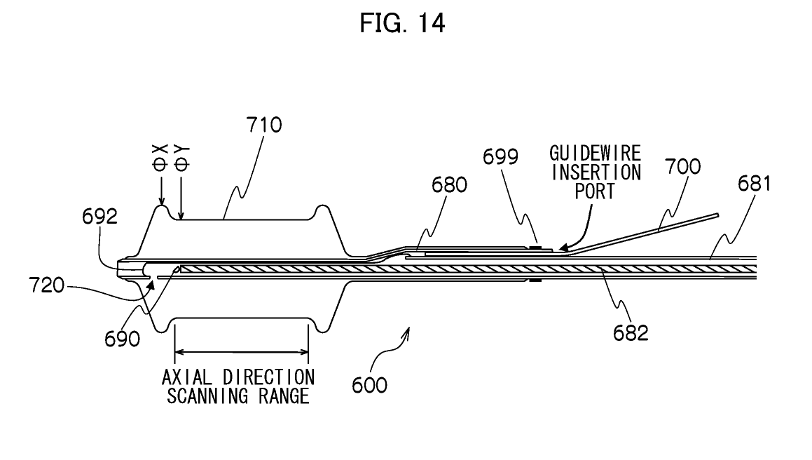

FIG. 14 is a cross-sectional schematic diagram of modification example 1 of the present embodiment. Modification example 1 illustrates a difference from the present embodiment. When the balloon 710 illustrated in the present embodiment is expanded at a fragile lesion part, there is a risk of damaging the lesion part. Therefore, according to modification example 1, a configuration is adopted such that a diameter φX at both ends of the balloon 710 is greater than a diameter φY at a center part thereof (φX>φY; for example, X=10 mm and Y=12 mm), a distance from the observation target is secured with the diameter φX at both ends of the balloon 710, and an area scanned by the ball lens 690 (axial direction scanning area) does not closely contact a lesion.

### Modification Example 2

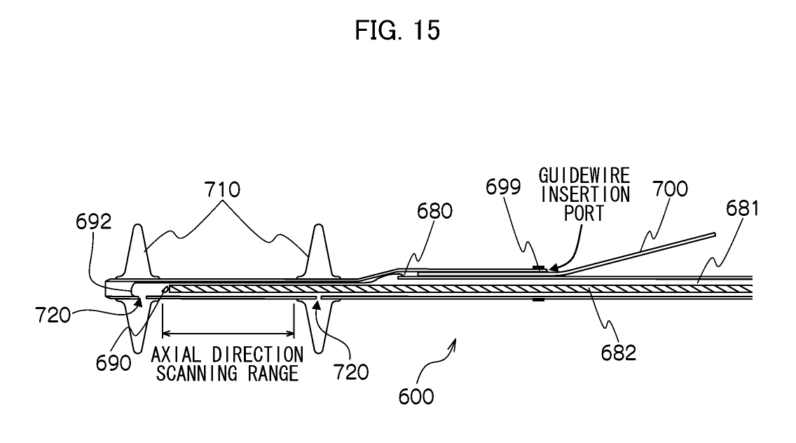

FIG. 15 is a cross-sectional schematic diagram of modification example 2 of the present embodiment. A difference with modification example 1 is that two independent balloons 710 are provided at the front and rear in the axial direction scanning range. Thus, since the balloons 710 are not present in the observation region (axial direction scanning range), there is no attenuation of the laser beam by the balloons 710, and observation is possible in a state in which a fixed distance from the observation target is maintained by the balloons 710.

### Modification Example 3

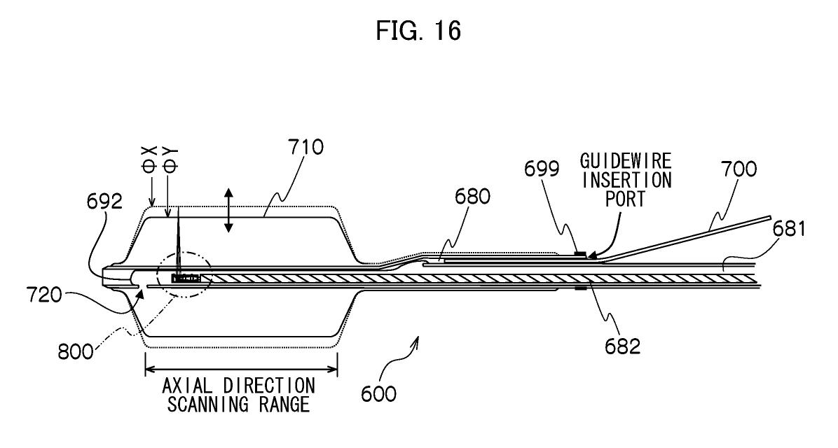

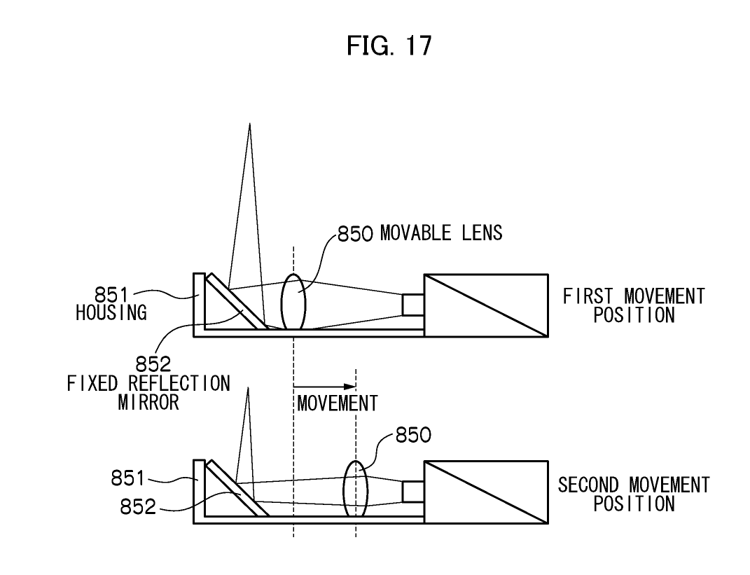

FIG. 16 is a cross-sectional schematic diagram of modification example 3 of the present embodiment. FIG. 17 is a view that shows an optical lens system at a distal end portion of an imaging core shown in FIG. 16. Modification example 3 describes a difference from the present embodiment. According to modification example 3, the balloon 710 has a compliance property, and the diameter thereof can be controlled by means of the pressurizing pressure. It is therefore possible to adjust a distance between a lesion part and the imaging core, and to observe an appropriate area. Further, an optical lens system 851 at the distal end portion of the imaging core does not have a fixed focus, but is configured so that, as shown in FIG. 17, by moving a movable lens 850 in the axial direction, the focus can be adjusted by means of a fixed reflection mirror 852 and the movable lens 850. It is therefore possible to adjust a focal distance in accordance with the expansion diameter of the balloon 710. Thus, by means of the OCT apparatus, an adjustment can be made so that the diameter of the balloon 710 and the focal distance are optimized simultaneously. The expansion diameter can be measured, for example, using the air-flow rate to the balloon 710 or the internal pressure of the balloon 710, or by image processing of an OCT image.

The optical probe of modification example 3 is an optical probe that includes an optical fiber and an optical component attached to a distal end portion of the optical fiber that are provided inside a sheath to be inserted into a body cavity, and that radiates light that is transmitted through the inside of the optical fiber towards biological tissue inside the body cavity by means of the optical component, in which the sheath includes: an imaging core lumen that houses the optical fiber having the optical component along a longitudinal axis; a balloon that is disposed so as to cover an outer side of the imaging core lumen; and a port for expanding/contracting the balloon by pressurization/depressurization that is at a proximal part of the sheath; and in which the optical component includes a focus adjustment mechanism, and a diameter of the balloon can be varied by a pressurizing pressure (the first configuration of modification example 3).

According to the configuration of the optical probe of modification example 3, in addition to the advantages of the present embodiment and the modification examples 1 and 2, there is the unique advantage that, by making the diameter of the balloon and the focal point of the optical system adjustable, observation can be performed over a wide area with the appropriate focus.

Further, according to the optical probe of modification example 3, with respect to the above described first configuration, preferably a focus obtained by the focus adjustment mechanism is controlled in accordance with the diameter of the balloon (the second configuration of modification example 3).

Further, according to the optical probe of modification example 3, with respect to the above described first or second configuration, preferably the sheath further includes a guidewire lumen that is disposed approximately parallel to a distal part of the imaging core lumen (the third configuration of modification example 3).

Furthermore, according to the optical probe of modification example 3, with respect to any one of the above described first to third configurations, preferably: the balloon is arranged so as to cover an outer side of the guidewire lumen and the imaging core lumen and is connected at one portion with the imaging core lumen; the imaging core lumen, the guidewire lumen, and the balloon are coaxially disposed at a most distal part; the port is connected to the imaging core lumen; the sheath further includes a flexible and optically transparent partition wall that separates the imaging core lumen and the guidewire lumen; by depressurizing the imaging core lumen, the partition wall is caused to block off the imaging core lumen, the most distal part is caused to function as a guidewire lumen, and the balloon is deflated; and by pressurizing the imaging core lumen, the partition wall is caused to block off the guidewire lumen, the most distal part is caused to function as an imaging core lumen, and the balloon is expanded (the fourth configuration of modification example 3).

### APPLICATION EXAMPLES

The OCT probe 600 of each of the foregoing embodiments can be used not only as a blood vessel catheter, but can also be applied to a diagnostic imaging apparatus in which the OCT probe 600 is used together with an endoscope apparatus.

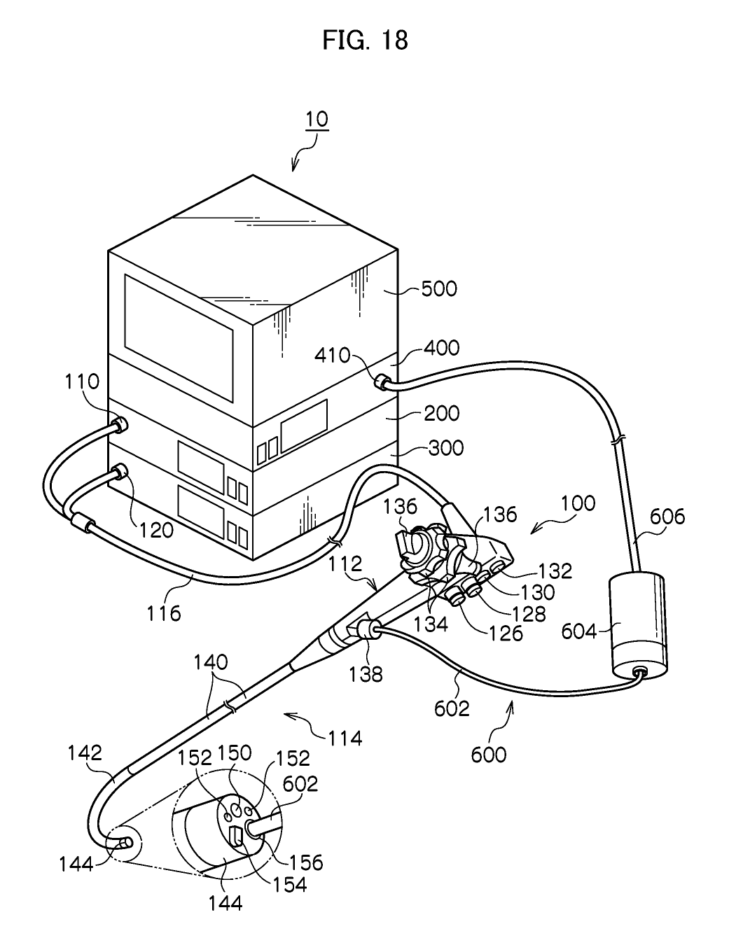

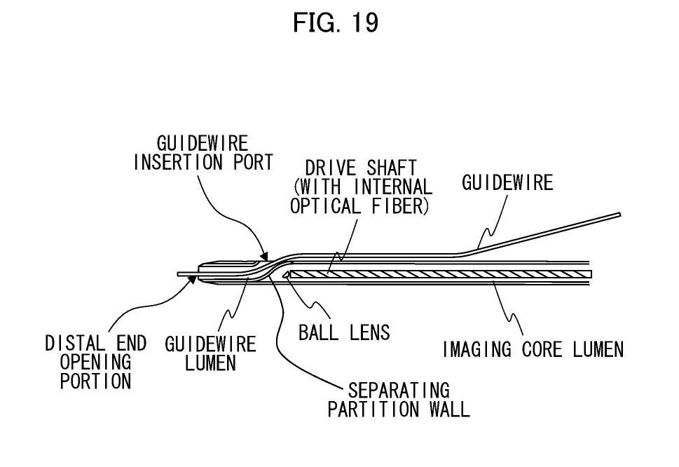

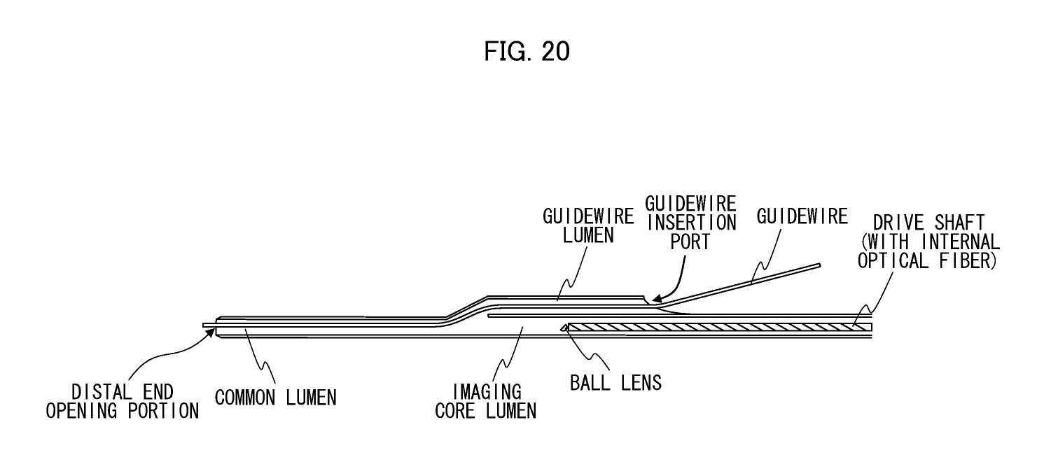

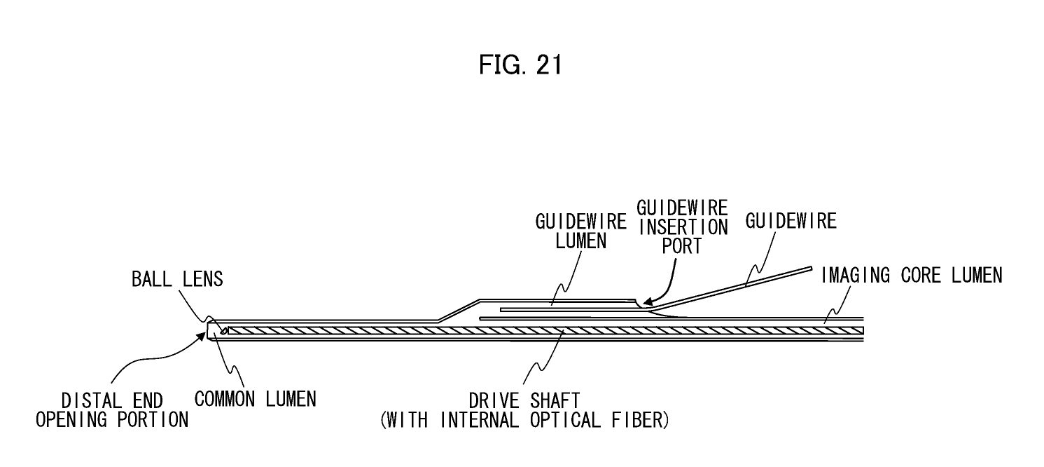

More specifically, as shown in FIG. 18, a diagnostic imaging apparatus 10 in which the OCT probe 600 of the present embodiment is used together with an endoscope apparatus mainly includes an endoscope 100, an endoscope processor 200, a light source apparatus 300, an OCT processor 400 as a living organism tomographic image generation apparatus, and a monitor apparatus 500 as a display device. The endoscope processor 200 may be configured so as to incorporate the light source apparatus 300.

The endoscope 100 includes a hand-side operation portion 112 and an insertion portion 114 that is connected to the hand-side operation portion 112. The physician grasps and operates the hand-side operation portion 112 and inserts the insertion portion 114 into the body of the subject to observe the inside of the body.

A forceps insertion portion 138 is provided in the hand-side operation portion 112. The forceps insertion portion 138 communicates with a forceps opening 156 of a distal end portion 144 via an unshown forceps channel provided inside the insertion portion 114. According to the diagnostic imaging apparatus 10, by inserting the OCT probe 600 as a probe from the forceps insertion portion 138, the OCT probe 600 can be led out from the forceps opening 156. The OCT probe 600 includes an insertion portion 602 that is inserted from the forceps insertion portion 138 and led out from the forceps opening 156, an operation portion 604 with which the physician operates the OCT probe 600, and a cable 606 that is connected to the OCT processor 400 via a connector 410.

An observation optical system 150, an illumination optical system 152, and a CCD (unshown) are provided in the distal end portion 144 of the endoscope 100.

The observation optical system 150 forms an image of a subject on a light-receiving surface of an unshown CCD. The CCD converts the image of the subject that has been formed on the light-receiving surface into electrical signals by means of respective light receiving elements. The CCD of the present embodiment is a color CCD in which color filters having the three primary colors red (R), green (G), and blue (B) are respectively arranged on each pixel in a predetermined array (a Bayer array, or a honeycomb array).

The light source apparatus 300 causes visible light to be incident on an unshown light guide. One end of the light guide is connected to the light source apparatus 300 via an LG connector 120, and the other end of the light guide faces the illumination optical system 152. The light emitted from the light source apparatus 300 passes through the light guide and is emitted from the illumination optical system 152 to illuminate the field-of-view area of the observation optical system 150.

An image signal output from the CCD is input to the endoscope processor 200 via an electrical connector 110. The analog image signal is converted into a digital image signal inside the endoscope processor 200, and is subjected to processing necessary for displaying the image signal on the screen on the monitor apparatus 500.

Thus, data of the observed image acquired by the endoscope 100 is output to the endoscope processor 200, and the image is displayed on the monitor apparatus 500 connected to the endoscope processor 200.

The optical probe and endoscope apparatus of the present invention have been described in detail above. However, it should be understood that the present invention is not limited to the above examples. Naturally, various improvements and modifications may be made to the invention within a range that does not depart from the spirit and scope of the present invention.

What is claimed is:

1. An optical probe that comprises an optical fiber and an optical component attached to a distal end portion of the optical fiber that are provided inside a sheath to be inserted into a body cavity, and that radiates a light that is transmitted through an inside of the optical fiber towards biological tissue inside the body cavity by means of the optical component,
wherein the sheath comprises:
an imaging core lumen that houses the optical fiber in a condition in which the optical fiber is movable forward or rearward along a longitudinal axis;
a guidewire lumen that is disposed approximately parallel to a distal part of the imaging core lumen;
a flexible and optically transparent partition wall that separates the imaging core lumen and the guidewire lumen; and
a pressure increasing/decreasing port for increasing/decreasing a pressure inside the imaging core lumen that is provided at a proximal part of the imaging core lumen.

2. The optical probe according to claim 1, wherein the imaging core lumen and the guidewire lumen are arranged along the longitudinal axis at a most distal part, the imaging core lumen is sealed at a portion other than the pressure increasing/decreasing port portion, and at least when the imaging core lumen is depressurized from the pressure increasing/decreasing port, the partition wall blocks off the imaging core lumen and the most distal part is caused to function as the guidewire lumen.

3. The optical probe according to claim 1, wherein the imaging core lumen and the guidewire lumen are arranged along the longitudinal axis at a most distal part, the imaging core lumen is sealed at a portion other than the pressure increasing/decreasing port portion, and at least when the imaging core lumen is pressurized from the pressure increasing/decreasing port, the partition wall blocks off the guidewire lumen and the most distal part is caused to function as the imaging core lumen.

4. The optical probe according to claim 1, wherein the imaging core lumen and the guidewire lumen are arranged along the longitudinal axis at a most distal part, the imaging core lumen is sealed at a portion other than the pressure increasing/decreasing port portion, and when the imaging core lumen is depressurized from the pressure increasing/decreasing port, the partition wall blocks off the imaging core lumen and the most distal part is caused to function as the guidewire lumen, and when the imaging core lumen is pressurized from the pressure increasing/decreasing port, the partition wall blocks off the guidewire lumen and the most distal part is caused to function as the imaging core lumen.

5. The optical probe according to claim 1, wherein the optical fiber is arranged inside a drive shaft that rotationally drives, and an inside of the body cavity is radially scanned by rotationally driving the optical component.

6. The optical probe according to claim 5, wherein the drive shaft is movable along the longitudinal axis, and an inside of the body cavity is spirally scanned by driving the optical component rotationally and in an axial direction.

7. The optical probe according to claim 1, wherein the optical component includes a ball lens having a reflective surface that bends, at approximately a right angle, a travelling direction of light that is transmitted through the inside of the optical fiber.

8. The optical probe according to claim 1, wherein the optical fiber transmits a wavelength-sweeping laser beam.

9. An endoscope apparatus that comprises an optical probe according to claim 1, wherein the sheath of the optical probe is inserted through a treatment instrument channel of an endoscope.

10. An optical probe that comprises an optical fiber and an optical component attached to a distal end portion of the optical fiber that are provided inside a sheath to be inserted into a body cavity, and that radiates a light that is transmitted through an inside of the optical fiber towards biological tissue inside the body cavity by means of the optical component, wherein the sheath comprises:
an imaging core lumen that houses the optical fiber in a condition in which the optical fiber is movable forward or rearward along a longitudinal axis;
a guidewire lumen that is disposed approximately parallel to a distal part of the imaging core lumen;
a balloon that is arranged so as to cover an outer side of the guidewire lumen and the imaging core lumen and that is connected at one portion to the imaging core lumen;
a flexible and optically transparent partition wall that separates the imaging core lumen and the guidewire lumen; and
a pressure increasing/decreasing port for increasing/decreasing a pressure inside the imaging core lumen that is provided at a proximal part of the imaging core lumen;
and wherein:
the imaging core lumen, the guidewire lumen, and the balloon are disposed along the longitudinal axis at a most distal part; and
the imaging core lumen is connected to the balloon and is sealed at a distal part, and depressurizing the imaging core lumen causes the partition wall to block off the imaging core lumen and causes the most distal part to function as the guidewire lumen and also deflates the balloon, and pressurizing the imaging core lumen causes the partition wall to block off the guidewire lumen and causes the most distal part to function as the imaging core lumen and expands the balloon.

11. The optical probe according to claim 10, wherein the optical fiber is arranged inside a drive shaft that rotationally drives, and an inside of the body cavity is radially scanned by rotationally driving the optical component.

12. The optical probe according to claim 10, wherein the drive shaft is also movable along the axial direction, and an inside of the body cavity is spirally scanned by driving the optical component rotationally and forward or rearward in an axial direction driving range.

13. The optical probe according to claim 10, wherein the optical component comprises a ball lens having a reflective surface that bends, at approximately a right angle, a travelling direction of the light that is transmitted through the inside of the optical fiber.

14. The optical probe according to claim 10, wherein the optical fiber transmits a wavelength-sweeping laser beam to the inside of the body cavity.

15. The optical probe according to claim 10, wherein a diameter at both ends of the balloon is larger than a diameter at a center part thereof.

16. The optical probe according to claim 10, wherein the imaging core lumen is connected to a plurality of balloons.

17. The optical probe according to claim 12, wherein the plurality of balloons are arranged at front and rear of the axial direction driving range.

18. An endoscope apparatus comprising an optical probe according to claim 10, wherein the sheath of the optical probe is inserted through a treatment instrument channel of an endoscope.

19. An optical probe that comprises an optical fiber and an optical component attached to a distal end portion of the optical fiber that are provided inside a sheath to be inserted into a body cavity, and that radiates a light that is transmitted through an inside of the optical fiber towards biological tissue inside the body cavity by means of the optical component, wherein the sheath comprises:
an imaging core lumen that houses the optical fiber having the optical component along a longitudinal axis;
a balloon that is disposed so as to cover an outer side of the imaging core lumen; and
a port for expanding/contracting the balloon by pressurization/depressurization that is at a proximal part of the sheath;
and wherein:
the optical component comprises a focus adjustment mechanism; and
a diameter of the balloon can be varied by a pressure that is applied.

20. The optical probe according to claim 19, wherein a focus obtained by the focus adjustment mechanism is controlled in accordance with a diameter of the balloon.

21. The optical probe according to claim 19, wherein the sheath further comprises a guidewire lumen that is disposed approximately parallel to a distal part of the imaging core lumen.

22. The optical probe according to claim 19, wherein:
the balloon is arranged so as to cover an outer side of the guidewire lumen and the imaging core lumen and is connected at one portion with the imaging core lumen;
the imaging core lumen, the guidewire lumen, and the balloon are coaxially disposed at a most distal part;
the port is connected to the imaging core lumen;
the sheath further comprises a flexible and optically transparent partition wall that separates the imaging core lumen and the guidewire lumen;
by depressurizing the imaging core lumen, the partition wall is caused to block off the imaging core lumen, the most distal part is caused to function as a guidewire lumen, and the balloon is deflated; and
by pressurizing the imaging core lumen, the partition wall is caused to block off the guidewire lumen, the most distal part is caused to function as an imaging core lumen, and the balloon is expanded.
